(19) United States
(12) Patent Application Publication
(10) Pub. No.: US 2006/0211940 A1
(43) Pub. Date: Sep. 21, 2006

(54) Blood vessel structure segmentation system and method

(76) Inventors: Marco Antonelli, Genova (IT); Silvana Delleplane, Genova (IT); Vadin Peretroukhine, Toronto (CA)

Correspondence Address: Dowell & Dowell, P.C.;Suite 406, 2111 Eisenhower Avenue, Alexandria, VA, 22314, US

(21) Appl. No.: 11/240,624

(22) Filed: Oct. 3, 2005

Related U.S. Application Data
(60) Provisional application No. 60/614,495, filed on Oct. 1, 2004.

(51) Int. Cl. A61B 5/05

(52) U.S. Cl. 600/410

(57) ABSTRACT

The invention relates to a system and method for segmenting an image of a plurality of structures stored as a set of spatially related data points. The data points represent variations in a predetermined parameter which allows the segmentation to occur. Once the data is acquired, a seed point is selected indicating a structure of interest. Each of the data points is assigned a value of connectivity as to the confidence that it is part of the same structure of the seed point. An endpoint is selected of the structure of interest and a path is built between the seed point and the end point based on the values of connectivity. Planes are cut along the path and a final connectivity is determined using the data points located on each plane thereby producing a final segmented image.

This application claims priority from U.S. provisional patent application No. 60/614,495 filed Oct. 1, 2004.

### FIELD OF THE INVENTION

The present invention relates to the field of imaging and in particular to a system and method for segmenting certain subsets of images in order to isolate structures. The invention has particular utility in the segmentation of blood vessel structures.

### BACKGROUND OF THE INVENTION

Many diseases are due to an imperfect working of the main human blood vessels; stenosis and aneurysms are only the major pathologies. At the state of the air, there are a substantial number of vascular diagnostic techniques, such as ultrasonic techniques, Digital Angiography, CT-Angiography (CTA) and others. Unfortunately, almost all angiographic techniques are very invasive. Some use X-ray, others require the injection of a contrast agent by using a probe placed very close to the district of interest.

In the last years the novel technique of Magnetic Resonance Angiography (MRA), in particular the Contrast-Enhanced version (CE-MRA), has been largely accepted by the medical community. In addition to having better quality of image compared to traditional angiography, one of the major benefits of this technique is that it is almost non-invasive. It is well known that Magnetic Resonance does not use ionizing radiation and the contrast agent used in this technique is less hazardous then the ones used in CTA.

CE-MRA can be acquired in two different acquisition modalities: dynamic and steady state. A dynamic acquisition provides a synchronization among acquisition time and contrast agent infusion. With a perfect timing the result volume only shows the artery structures enhanced. This acquisition requires an estimation of some non-measurable variables like the rate or the speed of blood flow. However, because of the high speed of the acquisition process, the acquired images have a low resolution. On the other hand, the steady state acquisition exploits the longer time persistence that distinguishes the contrast agents used in CE-MRA. This results in images that show, when enhanced, the complete structures of the blood vessels. The steady state acquisition modality foresees a time delay between the contrast agent infusion and the image acquisition. This time is useful to get a perfect blend between agent and blood. In opposition to the dynamic acquisition, steady state acquisition is much simpler and provides a good resolution.

One of the drawbacks of CE-MRA is its poor image resolution, which causes problems such as partial volume effect. Partial volume effect refers to a number of effects which occur due to the finite size of the spatial elements (pixels) used by the diagnostic technique, it may also be caused by movements of the patient during the CE-MRA procedure. For example, when two blood vessels run very near one another, one or more contact points may occur. Since in a CE-MRA only the blood can be seen because of the contrasting agent, when two blood vessels enter in contact, they appear to be connected, thus the point of contact often cannot be seen through the visual analysis of the original plane of view. Typical segmentation techniques do not distinguish blood vessels in contact with each other and this is true when using any contrasting agent.

Another drawback of CE-MRA is the non-homogeneity of the concentration of contrasting agent in the blood vessels. Often, the contrasting agent does not distribute uniformly in the blood with the result that the lighter pixels are located on the external border of the blood vessel while the pixels located in the centre of the blood vessels are somewhat darker.

The above mentioned drawbacks are the major causes of the failure of image segmentation algorithms.

It is therefore an object of the present invention to provide a system and method which obviates or mitigates the above mentioned disadvantages.

### SUMMARY OF THE INVENTION

In one aspect, the present invention provides a method of segmenting an image of a plurality of structures that are stored as a set of spatially related data points which represent variations in a predetermined parameter. The method begins by selecting a seed point within a structure to be segmented. For each of the data points, a preliminary value of connectivity is assigned which is indicative of the confidence that respective ones of the data points are part of the same structure as the seed point. An end point is then selected within the structure to be segmented and a sequence of data points between the seed point and the end point is defined based on points having the a preliminary connectivity values above a predetermined value. For each data point of the sequence, a set of points associated with the data point is determined. A final value of connectivity is then assigned to each data point in the sequence which is indicative of the confidence that respective points of said associated set of points are part of the same structure as the seed point and end point.

In another aspect, the present invention provides an imaging apparatus. The imaging apparatus has a data storage having a set of spatially related points representing variations in a predetermined parameter. The imaging apparatus also has a first comparator to compare a value of the predetermined parameter at the points with that of a seed point part of a structure and establish a preliminary value of connectivity which is indicative of the confidence that respective data points are part of the same structure as the seed point. The imaging apparatus also has a second comparator to compare the preliminary value of connectivity of a sequence of data points which connects the seed point to an end point of the structure with that of a set of points associated with each said data point. This final value of connectivity is indicative of the confidence that the data points in the sequence are part of the same structure as the seed point and the end point.

### BRIEF DESCRIPTION OF THE DRAWINGS

Embodiments of the invention will now be described by way of example only with reference to the accompanying drawings in which:

FIG. 1 is a schematic diagram depicting the components of a vascular diagnostic imaging system.

FIG. 2 is a schematic diagram depicting a stack of cross-sections forming a three-dimensional array of voxels.

FIG. 3 illustrates a generalized flow chart of an image segmentation algorithm.

FIG. 4 shows a graph of a characteristic function βa(v).

FIG. 5 illustrates a generalized flow chart of an algorithm to determine the connectivity of two voxels.

FIG. 6 shows a perspective view of two blood vessel structures.

FIG. 7 shows a perspective view of the two blood vessel structures of FIG. 6 as seen by a CE-MRA.

FIG. 8 shows a cross-sectional view (along axis VIII-VIII as shown in FIGS. 6 and 7) of the two blood vessel structures shown in FIGS. 6 and 7.

FIG. 9 shows a cross-sectional view (along axis IX-IX as shown in FIGS. 6 and 7) of the two blood vessel structures shown in FIGS. 6 and 7.

FIG. 10 shows a cross-sectional view (along axis X-X as shown in FIGS. 6 and 7) of the two blood vessel structures shown in FIGS. 6 and 7.

FIG. 11 shows a s-path applied to the blood vessel structures of FIG. 7.

FIG. 12 shows a perspective view of a s-path with associated normal planes.

FIG. 13 illustrates a generalized flow chart of an algorithm to determine the s-path based 2D connectivity of two voxels.

FIG. 14 shows a perspective view of a s-path with associated pairs of orthogonal planes.

FIG. 15 illustrates a generalized flow chart of an algorithm to determine the s-path based 2D connectivity of two voxels with associated pairs of orthogonal planes.

### DETAILED DESCRIPTION OF THE INVENTION

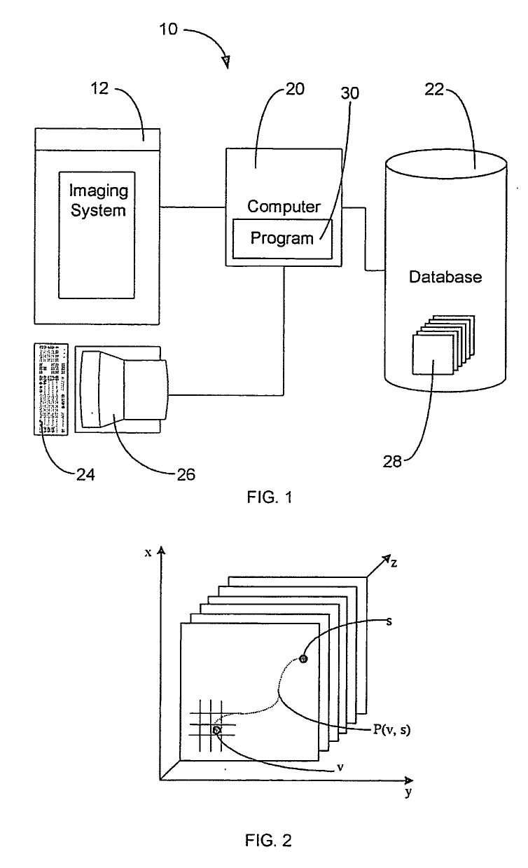

FIGS. 1 to 13 present a system and methodology for the segmentation of blood vessel structures, for example arteries and veins, from other structures and from each other, starting from a vascular diagnostic technique utilizing an imaging system. For illustrative purposes, the example described herein will refer to a system using a Contrast-Enhanced Magnetic-Resonance-Angiography (CE-MRA) due to its low level of invasiveness and thus is the most preferable method of vascular diagnosis incorporating the present invention. It will be appreciated that other vascular diagnostic imaging techniques may incorporate the teachings of the present invention and it is not intended to limit the system to only CE-MRA.

For example, incorporating an acceptable contrasting agent CTA would be a suitable substitute. Such application of the present invention would therefore enhance separation of structures imaged using any vascular diagnostic method. It will be appreciated that the methods and apparatus described herein are suitable for segmenting structures of any data set, e.g. bone structures, and reference to vascular segmentation is made for illustrative purposes only.

Referring to FIG. 1, a vascular diagnostic system for acquiring the image data of a subject, segmenting blood vessels structures from the image data and displaying such structures, is indicated generally at numeral 10.

The system 10 comprises an imaging system 12 and in this example a CE-MRA imaging system is used, to interrogate a patient having had a contrast agent injected into his or her bloodstream and supply data to a computer 20 from which an image can be created. The data is stored as a set of spatially related data points representing variations in intensity which can be displayed as variations in colour or grey scale. The computer 20 includes a program 30 for running on the computer, and to manipulate and display the data obtained from the CE-MRA imaging system. The program 30 comprises a set of machine readable instructions, which may be stored on a computer readable medium. Such a medium may include hardware and/or software such as, by way of example only, magnetic disks, magnetic tape, optically readable medium such as CD ROM's, and semi-conductor memory such as PCMCIA cards. In each case, the medium may take the form of a portable item such as a small disk, floppy diskette, cassette, or it may take the form of a relatively large or immobile item such as hard disk drive, solid state memory card, or RAM provided in the computer 20. It should be noted that the above listed example mediums can be used either alone or in combination.

The data and resultant images are stored on a database 22 and accessed via a user interface 24, such as a keyboard, mouse, or other suitable devices, for display on a display 26. If the display 26 is touch sensitive, then the display 26 itself can be employed as the user interface 24. Usually, during an imaging procedure, the CE-MRA imaging system 12 scans a patient, producing a series of cross-sectional images (or slices) of the patient's body. These cross-sectional images composed of pixels, each having a measurable intensity value, are then forwarded to the computer 20. The program 30 stacks the data in a three-dimensional array of voxels creating a three-dimensional image of the patient for viewing as a displayed image on display 26 and storing as a data-set 28 in the database 22. A voxel, or volume pixel, is a spatial element defined as the smallest distinguishable part of a three-dimensional image. The user interface 24 provides facility for an operator to interact with the system, and more particularly, for selecting areas of the display image 26 for identifying structures to be processed or to set various parameters of the system. The displayed images may be generated using any suitable software and/or hardware, such as maximum intensity projection (MIP) visualization software, e.g., Visualization Toolkit available from VTK, version 3.1.

The computer 20 uses the program 30 to process the data-set 28 to produce the required image in a manner, which is described in more detail below.

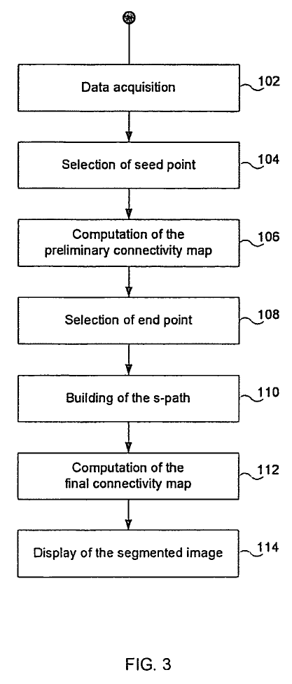

As shown in FIG. 2, typically each image is comprised of a stack of cross-sectional images forming a three-dimensional array made up of individual voxels v, which is stored as a data-set 28 in the database 22. The program 30 includes a segmentation algorithm which is depicted by the flow chart shown in FIG. 3. The sequence of steps composing the algorithm is indicated by the sequence of blocks 102 to 114. In block 102 the algorithm starts by taking the three-dimensional array as input and at block 104 selects a seed point, a, located in the structure of interest near one of its extremities. The seed point a is usually selected and entered into the system by the user using the user interface 24 to view the overall structure and select the area of interest.

At block 106, for each voxel v in the array, the algorithm calculates, as a preliminary definition of the object of interest, the connectivity between voxel v and the seed point a. This phase has two principal aims: perform a preliminary connectivity filtering and build a fuzzy connectivity tree of the structure of interest.

The connectivity from a specific voxel v to a seed point a is a function of the variation of a predetermined characteristic, such as voxel intensity, etc., along a path P(v, a) from the seed point a to the voxel v. Accordingly, a path P(v, a) is selected from the seed point a to the voxel v and the variation of the predetermined characteristic for each voxel along that path is determined. As will be described below, this variation is used to assign a value of connectivity to the voxel v.

The preliminary connectivity map, which depicts, for example, with higher grey levels the voxels that belong to the structure of interest, is then displayed to the user using the display 26 to view the overall structure and at block 108 the algorithm selects an end point, b, located in the structure of interest near the extremity opposite of the one where the seed point a is located. Similarly to the selection of the seed point, the end point b is usually selected and entered into the system by the user using the user interface 24 to view the overall structure and select the area of interest. Then, at block 110, the algorithm builds an s-path from seed point a to end point b. The s-path is the best internal path of the structure of interest, which may be defined as a connected sequence of voxels from seed point a to end point b having the highest connectivity values. During the calculation process of the preliminary connectivity map at block 106, all processed paths between seed point a and each voxel have already been computed, therefore it is a relatively simple matter to determine the s-path between seed point a and end point b. Although in this example, the voxels having the highest connectivity values are chosen, other criteria, such as the connectivity being of a predetermined value, above a particular threshold, or within a particular range etc., may also be used.

At block 112, the algorithm calculates the final connectivity map using s-path based 2D connectivity. The s-path based 2D connectivity may be seen as fuzzy filtering in order to discard nearby structures not fully connected to the structure of interest. This is based on the observation that contact points between two structures are usually not located along the whole length of each respective structure, but rather in relatively small localized areas. The principle of the s-path based 2D connectivity is that points of contact between two structures may be more easily seen in an alternative plane than the plane of data acquisition.

If it is assumed that the s-path is a good approximation of the skeleton of the structure of interest, then each point of the s-path may be used as a seed point for the s-path based 2D connectivity computation, which computes for each s-path seed point the connectivity between that seed point and all voxels located on a plane normal to the s-path at that seed point.

It should be noted that for the purpose of the s-path based 2D connectivity computation, typically only paths comprising points belonging to the normal plane are considered although other planes could be used with increased complexity. As will be described below, the s-path based 2D connectivity is used to assign a connectivity value to the voxels.

A second implementation uses two passes and, instead of planes normal to the s-path, a pair of planes with fixed orientation and orthogonal to each other are used. In the first pass the algorithm computes for each s-path seed point, the connectivity between that seed point and all voxels located on a plane P₁ which orientation is fixed for all seed point in the s-path (usually parallel to the XZ plane, since it doesn't involve costly computations of oblique planes) and containing that seed point. Again, only paths comprising points belonging to the P₁ plane are considered. In the second pass the algorithm computes for each s-path seed point the connectivity between that seed point and all voxels located on a plane P₂ orthogonal to P₁ (e.g. if P₁ is parallel to the XZ plane then P₂ can be parallel to the YZ plane) and containing that seed point. For each voxel the final connectivity value is taken as the minimum of the connectivity values assigned in the first and second passes.

It shall also be noted that the s-path may use filtering such as low-pass filtering. The s-path is not like the skeleton and it can be affected by some unwanted deviations. Therefore, some simplifications can be taken into account in order to reduce the computational complexity.

Finally, at block 114 the final connectivity map, which depicts, for example, with higher grey levels the voxels that belong to the structure of interest, is then displayed to the user using the display 26. The final connectivity map may also be used to create an MIP visualization for providing, e.g., a segmented image showing only the structure of interest or alternatively, a highlighted segmented portion and background data representing the remaining data points in the structure. It will be appreciated that any visualization techniques may be used and displayed in any way suitable to the application.

The connectivity may be determined in a number of different manners but a particularly beneficial one is to determine it mathematically, using fuzzy logic concepts. If the characteristic function βₐ(ν) over a fuzzy space, here either the three-dimensional array of voxels v composing the image being segmented in the case where the preliminary connectivity map is being computed or a subset of those voxels v defined by a specific plane in the case where the final connectivity map is being computed, assigns for the predetermined characteristic of each element v, a real value ranging in the interval [0,1] and the path P(ν, a) is a connected sequence of points from a voxel v to a voxel a, then the conventional fuzzy degree of connectedness C_{β} from v to a is expressed as follows:
C_{βa}(v)=conn(β_{a,}a, v)=max_{P(a, v)}[min_{pεP(a, v)}βₐ(p)] Equation 1
where C_{βa}(v) denotes the degree of connectedness, or connectivity, between v and a over characteristic function βₐ and P(a, v) is a path from a to v within the fuzzy space.

Thus the connectivity C_{β} is determined as the maximum of the minimum values of the predetermined characteristic in respective paths between the seed point a and the voxel v.

The characteristic function Pa takes into account the CE-MRA characteristics, which shows blood vessel structures with high intensity levels. The βₐ function privileges the voxel with the intensity that is higher than that of the seed point, in other words, the seed point along with any points having a higher intensity than that of the seed point have maximum membership and therefore are mapped with maximum grey level, this way, the highest intensity pixels are privileged. The βₐ function, for a voxel v and seed point a, may be defined as:

$$\begin{matrix}{β}_{a} = 1 + n ( v ) - n ( a ) if n ( v ) < n ( a ) \\ = 1 if n ( v ) \geq n ( a )\end{matrix} \\ where η ( v ) denotes intensity of voxel v . \qquad Equation 2$$

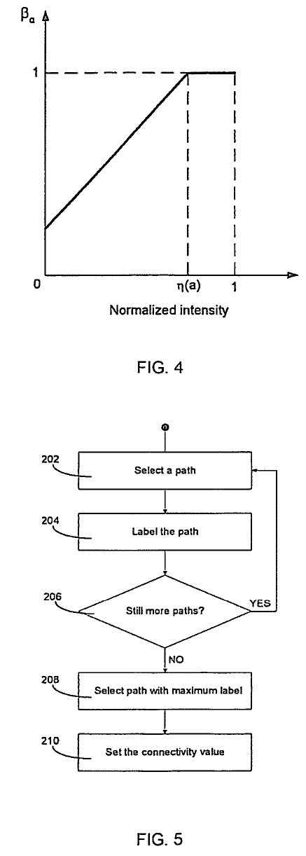

In FIG. 4, which graphically illustrates the above-defined βₐ function, it may be seen that all voxels v that have an intensity η(v) higher than the intensity of seed point a, η(a), are mapped with the best membership, i.e. 1, whereas the other are linearly rescaled.

The algorithm to obtain the connectivity of a voxel v to a seed point a is depicted by the flow chart shown in FIG. 5. The sequence of steps composing the algorithm is indicated by the sequence of blocks 202 to 210. In block 202 the algorithm starts by selecting an unvisited path, within the fuzzy space, from the seed point a to the voxel v. The selection of a path may be performed by any suitable algorithm although the algorithm described by Dellepiane et al. in “Nonlinear Image Labeling for Multivalued Segmentation”, IEEE Transactions on Image Processing, Vol. 5, No. 3, Mar. 1996, pp. 429-446, has been found to be particularly useful.

At block 204, the algorithm labels the selected path with the minimum voxel membership of all voxels in the path. At block 206 the algorithm determines whether all paths, within the fuzzy space, from the seed point a to the voxel v have been considered. If not the algorithm returns to block 202 in order to select another path. When all the paths have been visited, the algorithm then proceeds to block 208 where the path with the maximum label value is selected. Finally, at block 210 the connectivity between the voxel v and the seed point a is set as the label value of the selected path in block 208. It should be noted that the algorithm returns a connectivity value in the [0,1] interval but other scales may be used as well. The algorithm depicted by blocks 202 to 210 produces an output array which is called the preliminary connectivity map. This preliminary connectivity map is later used to determine the final connectivity map, which in turn is used to display, e.g. a segmented structure on the display 26 using visualization software.

Therefore, the preliminary connectivity map can be used to assign a particular intensity value or greyscale value to voxels within a structure for displaying on the display 26 using any suitable imaging application. The segmented structure can be highlighted, isolated, outlined etc. Alternatively, a line along the path may overlay the displayed image, in order to identify the structure. The values of the connectivity map can also be used for quantitative analysis, e.g., measuring the narrowness or bulging of a vein, and therefore, the connectivity map need not be displayed.

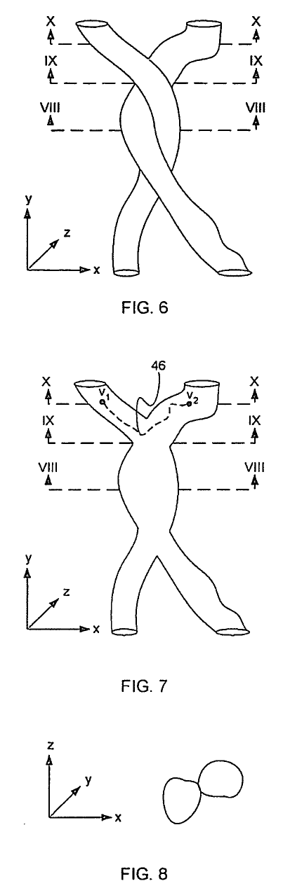

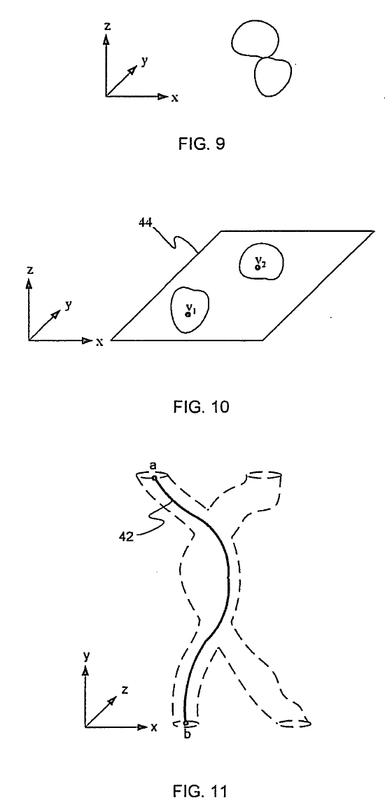

As previously mentioned, the principle of the s-path based 2D connectivity is that points of contact between two structures may be more easily seen in an alternative plane than the plane of data acquisition. When two intertwining structures, such as shown in FIG. 6, are visualised using a CE-MRA, there is a risk that the two structures appear as though they form a single structure, such as illustrated by FIG. 7. This is caused by the fact that in the CE-MRA only the contrasting agent in the blood stream is seen and of the partial volume effect, which is due to the finite size of the voxel (resolution) and the relative thinness of the structures under observation as well as slight displacements of those structures. Referring back to FIG. 7, the points of contact between the two structures cannot be distinguished in the original plane of acquisition, they may be more easily seen in alternative planes, such as illustrated by FIGS. 8 and 9.

Thus, the s-path based 2D connectivity introduced at block 112 uses each point composing the s-path 42, illustrated in FIG. 11, as a seed point from which a fuzzy space is defined. If SP is the set of seed points, then we have:
SP={∀sᵢ∈s-path(b, a)} Equation 3

where sᵢ represents the i^{th} point on the s-path s, and path (b,a) represents the path from seed point a to end point b.

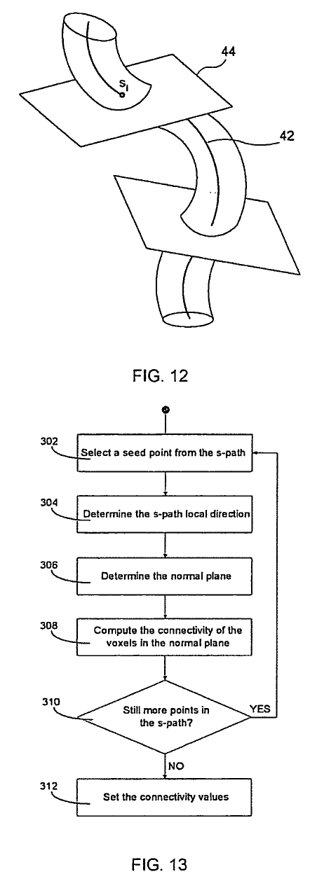

If the seed points sᵢ ε SP, then the s-path 42 local direction θs-path (sᵢ,SP) is defined by the following formula:
\overline{θ}ₛ₋ₚₐₜₕ(sᵢ,SP)=vector(s_{i-w}, . . . , s_{i+w}) Equation 4
with w ε N wherein N defines an optimal window value. N is a positive integer which defines how many adjacent points on the s-path are used to calculate the local direction of the path. For example, if the current point on the path is indexed as 10 (i=10) and the optimal window has been designated as w=3, then the direction of the s-path at the point i=10 is calculated using the points indexed as 7, 8, 9 and 11, 12, 13 (e.g. the 3 preceding points and the 3 following points). Vector (s_{i-w, . . . , s}_{i+w}) is a function that returns a normal vector to the s-path 42 and passing by point s; from which a plane 44 normal to the s-path 42 may be defined, as illustrated in FIG. 12.

Therefore, if C2D (volume, seed point, normal vector) is the bi-dimensional version β° of C_{βa}, the final output indicating a value of connectedness C, may be expressed as follows:

$$C = {{⋃}^{num [ SP ]}}_{i = 0} {C}_{{β}_{z}}^{2 D} ( {C}_{{β}_{z}} , {s}_{i} , {\overline{θ}}_{s - paht} ( {s}_{i} , SP ) ) \qquad Equation 5$$

points such as V₁, and V₂ of FIG. 7, which used to be mapped onto the same structure since in the original volume space there existed a path 46 connecting them, are now segregated since there are no paths connecting them in the plane 44 normal to the s-path 42, such as illustrated in FIG. 10.

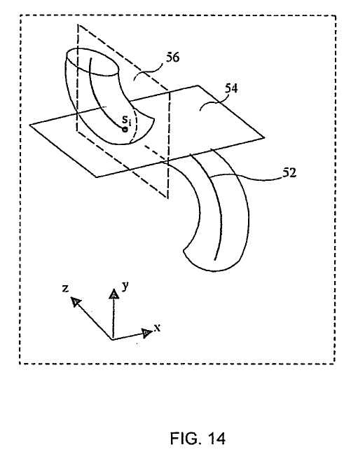

The algorithm to perform the s-path based 2D connectivity is depicted by the flow chart shown in FIG. 13. The sequence of steps composing the algorithm is indicated by the sequence of blocks 302 to 312. In block 302 the algorithm starts by selecting a seed point from the s-path which was built at block 110 of the FIG. 5 flow chart. The algorithm then determines, at block 304, the s-path local direction at the previously selected seed point from block 302. From this s-path local direction, a normal plane to s-path is defined at block 306, following which, at block 308, the connectivity value, or 2D connectedness, is computed for each of the voxels included in the normal plane to the selected seed point using the previously obtained preliminary connectivity value of each voxel. At block 310 the algorithm determines whether all points of the s-path have been considered. If not the algorithm returns to block 302 in order to select another point as a seed point. When all the s-path points have been processed the algorithm then proceeds to block 312 where the connectivity values are set. It should be noted that the algorithm returns connectivity values in the [0,1 ] interval but other scales may be used as well. The algorithm depicted by blocks 302 to 312 produces an output array which is the final connectivity map, which is used for displaying, e.g., a segmented structure or connectivity mapping on display 26. The output array would therefore be used to, e.g., assign voxel intensities or to determine an outline for highlighting the segmented structure in the image.

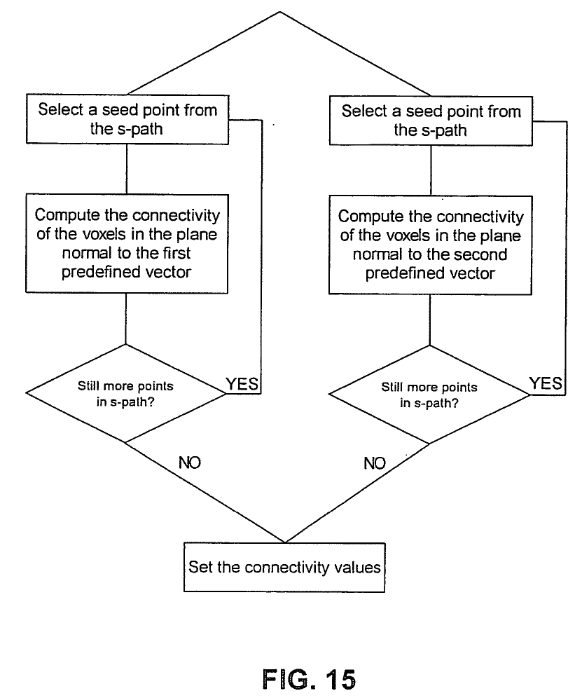

FIG. 14 illustrates the second implementation in which the algorithm uses a pair of orthogonal planes instead of one normal plane. This variation of the algorithm is also depicted by the flow chart shown in FIG. 15. Note that the Equations 1,2 and 3 are also applicable in this case and the Equation 5 can be modified and shall be denoted Equation 5′as follows:

$$C = {{⋃}^{num [ SP ]}}_{i = 0} min ( {C}_{{β}_{a}}^{2 D} ( {C}_{{β}_{a}} , {s}_{i} , {Θ}_{1} ) , {C}_{{β}_{a}}^{2 D} ( {C}_{{β}_{a}} , {s}_{i} , {Θ}_{2} ) ) , \qquad Equation 5$$

where Θ₁ is orthogonal to Θ₂.

Referring back to FIG. 15, in the first pass, a seed point is selected from the s-path in step 402, the connectivity of the voxels in the plane normal to the first predefined vector is computed in step 404 and a decision criteria 406 checks whether or not there are more points in the s-path where if the answer is “yes” then step 402 repeats. Similar steps occur during the second pass in steps 408, 410 and 412 respectively. When there are no remaining points in the s-path, the connectivity values are set in step 412 and for each voxel the final connectivity value is taken as the minimum of the connectivity values assigned in the first and second passes. The final connectivity values may then be used for display purposes as discussed above.

In yet another embodiment, the present invention may incorporate multiple pairs of seeds thereby segmenting branches in a vascular structure piece by piece. Alternatively the present invention may be used to target specific areas of interest using ordinary segmentation methods for the other areas (e.g. branches of veins and arteries) to extract images of the complete vascular structure beyond just a single artely of interest. Other structures, such as bone structures may also be segmented using the principles discussed above.

Although the present invention has been described by way of a particular embodiment thereof, it should be noted that modifications may be applied to the present particular embodiment without departing from the scope of the present invention and remain within the scope of the appended claims.

What is claimed is:

1. A method of segmenting an image of a plurality of structures stored as a set of spatially related data points representing variations in a predetermined parameter, said method comprising the steps of:
selecting a seed point within a structure to be segmented,
assigning to each of the data points a preliminary value of connectivity indicative of the confidence that respective ones of the data points are part of the same structure as said seed point,
selecting an end point within the structure to be segmented,
defining a connected sequence of data points having a preliminary connectivity value above a predetermined value, starting with said seed point and ending with said end point,
defining for each data point of said connected sequence of data points an associated set of points, and
assigning to said each data point of said connected sequence of data points a final value of connectivity indicative of the confidence that respective points of said associated set of points are part of the same structure as said seed point and said end point.

2. The method of claim 1 wherein said associated set of points define a plane passing through the respective data point of said connected sequence of data points.

3. The method of claim 2 wherein each said plane is normal to a respective vector passing through said respective data point of said connected sequence of data points.

4. The method of claim 2 wherein said associated set of points defines a pair of orthogonal planes passing through the respective data point of said connected sequence of data points, and said final values of connectivity are indicative of the confidence that respective minimum connectivity values of values assigned in an initial pass along a first of said pair of orthogonal planes and a second pass along a second of said pair of orthogonal planes are part of the same structure as said seed point and said end point.

5. The method of claim 1 further comprising the step of displaying said connected sequence of data points for illustrating either said preliminary or said final values of connectivity.

6. The method of claim 1 wherein said preliminary values of connectivity are determined according to a function of the variation of a predetermined characteristic along a path from said seed point to the respective data point.

7. The method of claim 1 wherein the determination of said final values of connectivity comprises discarding nearby structures not fully connected to said structure to be segmented.

8. The method of claim 1 wherein the determination of said final values of connectivity comprising filtering said data points.

9. The method of claim 1 wherein said final value of connectivity is determined according to the following steps:
for each data point after said seed point, determining a local direction from the preceding data point to the current data point;
determining said plane wherein said plane is normal to said local direction;
computing a value of connectivity for each data points in said area on said plane; and
assigning said final value of connectivity for said data point based on the values of connectivity for said data points in said area.

10. An imaging apparatus comprising:
a data storage having a set of spatially related data points representing variations in a predetermined parameter,
a first comparator to compare a value of said predetermined parameter at said data points with that of a seed point part of a structure and establish a preliminary value of connectivity indicative of the confidence that respective ones of said data points are part of the same structure as said seed point, and
a second comparator to compare said preliminary value of connectivity of a sequence of said data points connecting said seed point to an end point part of said structure with that of a set of points associated with respective ones of said data points to establish a final value of connectivity indicative of the confidence that respective ones of said data points are part of the same structure as said seed point and said end point.

11. The apparatus of claim 10 wherein said associated set of points define a plane passing through the respective data point connecting said seed point to an end point.

12. The apparatus of claim 10 further comprising a display for displaying a mapping of said data points to show said preliminary and final values of connectivity.

13. The apparatus of claim 10 wherein each said comparator further comprises a filter for filtering said data points.

14. The apparatus of claim 10 wherein each said comparator chooses a plane normal to a vector passing through each said data point.

15. The apparatus of claim 10 wherein each said comparator comprises a fuzzy logic module for performing fuzzy logic concepts in determining said preliminary and final values of connectivity.
